(19) United States
(12) Patent Application Publication
(10) Pub. No.: US 2025/0295305 A1
(43) Pub. Date: Sep. 25, 2025

(54) METHOD AND APPARATUS FOR MANAGEMENT OF EYE HEALTH

(71) Applicant: Sina Fateh, Santa Clara, CA (US)

(72) Inventor: Sina Fateh, Santa Clara, CA (US)

(21) Appl. No.: 19/228,059

(22) Filed: Jun. 4, 2025

Related U.S. Application Data
(63) Continuation-in-part of application No. 18/531,224, filed on Dec. 6, 2023, now Pat. No. 12,370,120.
(60) Provisional application No. 63/528,868, filed on Jul. 25, 2023.
(60) Provisional application No. 63/655,921, filed on Jun. 4, 2024.

(51) Int. Cl. A61B 3/028; A61B 3/00

(52) U.S. Cl. CPC A61B 3/028; A61B 3/0041

(57) ABSTRACT

A retinal map with visual regions provides a description of retinal health/function. A therapeutic light map with visual emphasis regions provides a prescription for modifications for light delivered to the retina to improve health. A filtering light map with display emphasis regions provides modifications to the light generated by a display. Correspondence among the retinal map, therapeutic light map, and filtering light map facilitate changes in light output by the display, such that light from the filtering light map on the display is delivered to the retina in accordance with the therapeutic light map, biasing the eye away from myopia or towards some other therapeutic aim. Thus an evaluation of eye health may be transformed into suitable modifications for pixel values on the display, so as to improve eye health. Display control may be fine grained, even to individual pixel by pixel control of the entire display.

### CROSS REFERENCE TO RELATED APPLICATIONS

This application claims the benefit of priority of U.S. Provisional Patent Application 63/655,921, filed 4 Jun. 2024, and is a continuation-in-part of U.S. patent application Ser. No. 18/531,224, filed 5 Dec. 2023, which claims the benefit of priority of U.S. Provisional Patent Application 63/528,868, filed 25 Jul. 2023, the contents of each of which are hereby incorporated by reference in their entireties, for all intents and purposes.

### FIELD

Various embodiments concern treatments for addressing myopia and diagnostic support related thereto. More particularly, various embodiments relate to non-pharmaceutical approaches for reducing, halting, and/or reversing the progression of myopia.

### BACKGROUND

Stresses of various sorts in or on the eyes may be associated with certain medical conditions, and/or resultant symptoms of those conditions. For example, certain forms of myopia may be caused or at least aggravated by the use of visual displays such as those found on smart phones, laptops, and desktop computers. For example, a smart phone display may typically present bright, high contrast content to a viewer's central vision at a relatively short focal distance (e.g., significantly less than an arm's length), while some or all of the viewer's peripheral vision does not receive content from the display and instead receives visual input from the surrounding environment which may be dark, low contrast, and/or at a longer focal distance. Such differences between what is received by the eye's central and peripheral vision may be interpreted by the body as a dysfunction of the eye, prompting neural adaptation (such as changes in the way the optic nerve and/or brain process information) and/or physical adaptation (such as changes in the shape of the retina and/or the eye as a whole).

Pharmaceutical approaches to treat the above may provide some useful results in at least certain cases. For example, atropine eye drops may be useful in counteracting certain forms of myopia to at least some degree. However, the mechanism by which atropine accomplishes such a function is not well understood, making it difficult to determine a suitable dose for a given patient, to identify contraindications, etc. In addition, while occasional use of atropine (e.g., for medical exams) has a long history and appears to be at least largely safe from a medical standpoint, the side effects, development of tolerance, potential for damage, etc. of long term, regular use remain unclear.

Furthermore, the structure and function of the human eye are extremely complicated and highly variable, with significant differences from group to group and even individual to individual, multiple biological processes that may interact in complex ways, etc.; consequently the precise stimuli as may lead to myopia (or other conditions) may be difficult to predict with a simple, generalized model. For example, a broad approach to decrease screen brightness to avoid changes leading to myopia may be effective for certain individuals, but may not necessarily be effective (and indeed may even be counterproductive) for other individuals. Thus, simple “one size fits all” behavioral approaches may not be widely effective, and may even be at least potentially harmful.

In addition, due to normal growth and/or ongoing development of myopia (and/or some other condition) the most effective parameters for assisting a given individual may vary over time. Consequently, relatively “fixed” systems for addressing myopia or myopia progression such as image altering optics (e.g., specialized glasses) may not be sufficiently adjustable to address variable and/or time changing conditions.

Even within a given eye at a particular time, considerable variation may exist in eye health, retinal functionality, etc. Thus, pharmaceutical approaches that affect the eye broadly, and approaches based on physical optics as likewise may affect the eye on a large scale, may suffer from insufficient potential for customization and inadequate “granularity” in addressing problems. For example, if a visual problem affects only certain portions of a retina, or certain portions more than others, then an approach that considers the retina only as a whole may be inefficient, unsuccessful, or potentially even counterproductive in at least certain instances.

### SUMMARY

This disclosure contemplates a variety of systems, apparatus, methods, and paradigms for addressing eye health concerns including but not limited to a progression of myopia, and for informing diagnosis, prescription, and evaluation related to such.

In a first aspect, a method is provided that includes determining a retinal map of a retina with the retinal map having visual regions therein, and determining a therapeutic light map responsive to the retinal map with the therapeutic light map having visual emphasis regions therein and a correspondence with the retinal map. The method includes determining eye orientation with respect to a display, and determining a filtering light map responsive to the therapeutic light map with the filtering light map having display emphasis regions therein and a correspondence with the therapeutic light map. Each display emphasis region exhibits at least one display emphasis to the display light output from the display.

The method includes applying the filtering light map to the display so as to apply the display emphases for the display emphasis regions to the display light output therefrom, and maintaining over time the eye orientation determination, the therapeutic light map, and the filtering light map. The method also includes maintaining over time the correspondence of the filtering light map with the therapeutic light map and the correspondence of the therapeutic light map with the retinal map such that light input delivered to the retina from the display as emphasized by the filtering light map corresponds with the therapeutic light map, the light input delivered to the retina biases the eye towards a therapeutic outcome, and the therapeutic outcome includes reversing myopia, preventing myopia, and/or slowing progression of myopia.

At least a portion of the display emphasis regions may each address one individual pixel, such that applying the filtering light map includes applying the display emphases for those portions of those display emphasis regions to those individual pixels. The display emphases for the display emphasis regions may individually address pixels in the display emphasis regions. The display emphases for said display emphasis regions may individually address selected pixels in the display emphasis regions. The selected pixels may be selected based on a pixel color value thereof.

The display may be non-transparent. In some embodiments, the display is transparent, and in other embodiments the display is non-transparent.

The retinal map may be determined at least in part through perimetry. The retinal map may be determined at least in part through measuring retinal sensitivity in the plurality of visual regions of the retina. The retinal map may be determined at least in part by performing a Humphrey visual field test. The retinal map may be determined at least in part by determining a deviation from the nominal geometry of the retina.

The retinal map may be determined at least in part by determining an eye geometry model of the eye, determining the therapeutic light map responsive to the eye geometry model, and determining the filtering light map responsive to the eye geometry model.

The display emphases for the display emphasis regions may include a change to any or all of the emphasis properties of brightness, contrast, saturation, resolution, hue, tint, display refresh rate, video frame rate, animation speed, focus, blur, sharpness, and diffusion.

The display emphases for the display emphasis regions may include a change to any or all of: the red channel, green channel, blue channel in a pixel color value; the red channel but not the green channel and the blue channel in said pixel color value; and the blue channel but not the red channel and the green channel in the pixel color value.

The display emphases for the display emphasis regions may include for an RGB color system a change to any one of a red, green, and blue color channel but no change to the other two; or a change to any two of red, green, and blue color channels but not the third. The display emphases for the display emphasis regions may include some or all color channels.

The therapeutic light map may be variable in time. The filtering light map may be variable in time, such that for the display emphasis regions at least a portion of the display emphases thereof are variable in time.

The method may include modifying the therapeutic light map responsive to the display light output. The method may include modifying the therapeutic light map responsive to the use of the display by the subject. The method may include modifying the therapeutic light map responsive to the display content of the display.

In a second aspect, an apparatus for biasing an eye of a subject toward a therapeutic outcome is provided that includes a display, an eye orientation monitor, and a processor. The processor has executable instructions instantiated thereon adapted to determine a therapeutic light map responsive to a retinal map of the retina with that includes visual regions of a retina of said eye, the therapeutic light map having visual emphasis regions and exhibiting a therapeutic correspondence the retinal map.

The executable instructions are adapted to determine an eye orientation for the eye with respect to said display in cooperation with the eye orientation monitor. The executable instructions are also adapted to determine a filtering light map for the display with the filtering light map including display emphasis regions and exhibiting a filtering correspondence with the therapeutic light map, responsive to the therapeutic light map and with respect to the eye orientation, with each display emphasis region exhibiting at least one display emphasis to the display light output from the display.

The executable instructions are also adapted to apply the filtering light map to the display so as to apply the display emphases for the display emphasis regions to the display light output, maintain over time the eye orientation determination, maintain over time the therapeutic light map and the filtering light map, and maintain over time the filtering correspondence of the filtering light map with the therapeutic light map so as to maintain display emphasis to the display light output for the display emphasis regions and maintain over time the therapeutic correspondence of the therapeutic light map with the retinal map, such that light input delivered to the retina from the display as emphasized by the filtering light map corresponds with the therapeutic light map, light input delivered to the retina biases the eye towards a therapeutic outcome, and the therapeutic outcome includes reversing myopia, preventing myopia, and/or slowing progression of myopia.

In a third aspect, an apparatus for biasing an eye of a subject toward a therapeutic outcome is provided that includes means for determining a retinal map of a retina with the retinal map having visual regions, means for determining responsive to the retinal map a therapeutic light map having visual emphasis regions and exhibiting a therapeutic correspondence with the retinal map, and means for determining eye orientation for the eye with respect to a display. The apparatus includes means for determining responsive to the therapeutic light map and with respect to the eye orientation a filtering light map for the display with the filtering light map having display emphasis regions and exhibiting a filtering correspondence with the therapeutic light map, each display emphasis region exhibiting at least one display emphasis to the display light output from the display.

The apparatus includes means for applying the filtering light map to the display so as to apply the display emphases for the display emphasis regions to the display light output therefrom, means for maintaining over time the eye orientation determination, means for maintaining over time the therapeutic light map and the filtering light map, and means for maintaining over time the filtering correspondence of the filtering light map with said therapeutic light map so as to maintain the at display emphasis to the display light output for the display emphasis regions and for maintaining over time the therapeutic correspondence of the therapeutic light map with the retinal map, such that light input delivered to the retina from the display as emphasized by the filtering light map corresponds with the therapeutic light map, the light input delivered to the retina biases the eye towards a therapeutic outcome, and the therapeutic outcome includes one or more of reversing myopia, preventing myopia, and slowing the progression of myopia.

### BRIEF DESCRIPTION OF THE SEVERAL VIEWS OF THE DRAWINGS

Various objects, features, and characteristics will become more apparent to those skilled in the art from a study of the following Detailed Description in conjunction with the appended claims and drawings, all of which form a part of this specification. While the accompanying drawings include illustrations of various embodiments, the drawings are not intended to limit the claimed subject matter.

FIG. 01 shows an example illustration of light focusing in an eye in cross section, with focusing at the retina.

FIG. 02 shows another example illustration of light focusing in an eye in cross section, with focusing in front of the retina.

FIG. 03 shows an example illustration of a retina with a retinal map defined with respect thereto.

FIG. 04 shows an example illustration of a therapeutic light map.

FIG. 05 shows an example illustration of a display device with a filtering map applied thereto.

FIG. 06 shows an example method for applying a visual emphasis to provide a therapeutic bias, in flow chart form.

FIG. 07 shows an example apparatus for applying a visual emphasis to provide a therapeutic bias.

FIG. 08 illustrates an example of a computer network system, in which various embodiments may be implemented, in schematic form.

The figures depict various embodiments described throughout the Detailed Description for the purposes of illustration only. While specific embodiments have been shown by way of example in the drawings and are described in detail below, the technology is amenable to various modifications and alternative forms. The intention is not to limit the technology to the particular embodiments described. Accordingly, the claimed subject matter is intended to cover all modifications, equivalents, and alternatives falling within the scope of the technology as defined herein.

### DETAILED DESCRIPTION

As an initial and non-limiting explanation, regional emphasis may be carried out for therapeutic effect by applying a differential in visual emphasis to a viewer's eyes, by applying a differential in display emphasis so as to produce the desired visual emphasis differential. Such an approach may also be understood as providing a selective digital image filter that acts on one or more specific areas of visual content representing different areas of the retina. More colloquially, a retina may be mapped for health, functionality, other concerns, etc. Changes in light input as may be beneficial to the retina based on the retinal map may then be identified, and may be understood as a therapeutic map. Changes then may be made to the output of a display, in a map of such changes applied to that display, so that the light from that display that reaches the retina is modified according to the therapeutic map.

Thus, the retina may be mapped, beneficial changes to light may be identified, and changes to light output from a display may be implemented on that display (e.g., in software controlling the display) to provide those beneficial changes in light at the retina, and in turn to provide any therapeutic benefits thereof to the eye.

Such an approach may be made with as few as one or two regions, e.g., dimming or brightening one region in comparison to another, changing color saturation in one region compared to another, etc. However, much more sophisticated approaches also may be implemented. A retina may be mapped in considerable detail, with dozens to hundreds of visual regions (or more) identified thereon. A therapeutic map may then be determined so as to provide changes emphasizing (e.g., increasing/decreasing) light level, color saturation, etc. as to be delivered to the retina also on a fine grained level with many visual emphasis regions in such a therapeutic map. In order to deliver such visual emphasis with light input to the retina, a display that the subject is looking at may then have some or all of the display area thereof modified according to a filtering map, with potentially many individual display emphasis regions in the filtering map. The filtering map may be applied to whatever output the display is presenting, whether text, video, images, animations, etc. (typically though not necessarily by a processor controlling the display). Thus the actual light output from the display may be altered from a default value through application of the filtering map, in any or all of the individual display emphasis areas thereof.

Considered somewhat differently, certain approaches herein may convert retinal deficits or other features into a retinal map form, e.g., a grid as may have pixel values for each visual region therein. Suitable compensation for those deficits, etc., may be determined in the form of a therapeutic map, e.g., a grid as may have digital compensation values for each visual emphasis region therein. A filtering map for a display then may be determined, e.g., a grid as may have digital values for altering (emphasizing, whether positively or negatively) the display output in the display emphasis regions thereof. Retinal features may be converted into some form of pixel values, and digital compensation determined and applied thereto. At least potentially, such operations may be carried out on the level of control some or even all individual pixels, even for very large arrays of pixels.

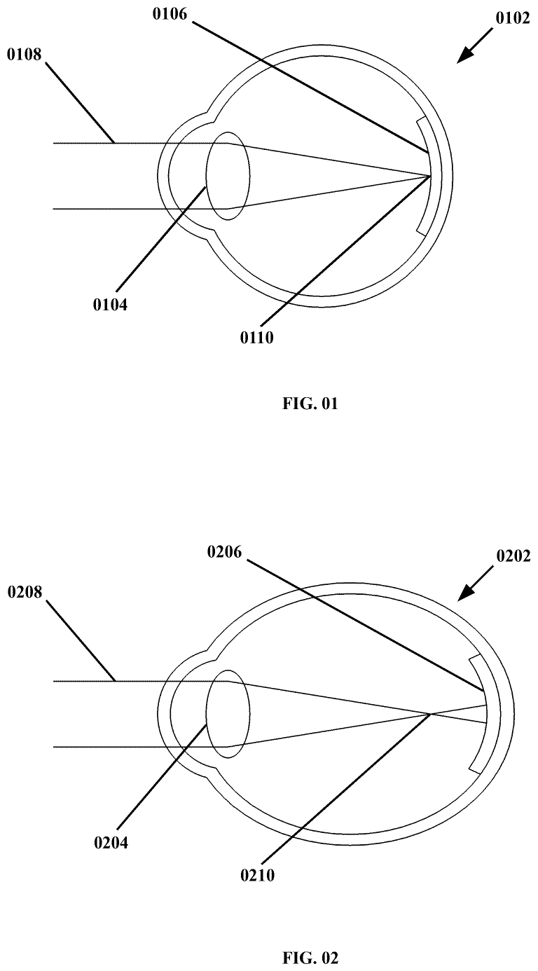

Now with reference to FIG. 01, a representation of an eye 0102 is presented there in cross section. As may be seen the eye 0102 includes a lens 0104 that focuses light and a retina 0106 that detects light. Example light input 0108 is shown entering the eye 0102, being converged by the lens 0102, and reaching a point of focused image 0110 wherein the image is properly focused for viewing at the surface of the retina 0106. It is noted that FIG. 01 is illustrative and is not intended to be precisely accurate in a physical or biological sense. In practice light typically does not reach only a single point of the retina, nor is light necessarily focused into a point at all. Likewise the structure of the eye generally and elements thereof may be substantially more complex than is shown. However, given the arrangement in FIG. 01, it may be understood that light input 0108 is being received at the retina 0106 in such a way as to be properly in focus for viewing, so that the subject perceives a clear image.

Now with reference to FIG. 02, another representation of an eye 0202 is presented there in cross section. However, as may be seen the eye 0202 in FIG. 02 is significantly distorted, e.g., exhibiting an increased axial length compared to the eye 0102 in FIG. 01. For certain medical issues, notably forms of myopia, eyes may in fact exhibit an increased axial length as visible in FIG. 02. The degree of increase in length visible in FIG. 02 may not represent that as typically may be seen in practice, and is shown for informative purposes rather than necessarily for biological precision. Regardless, as may be seen in FIG. 02 the eye 0202 therein again includes a lens 0204 and a retina 0206. However, when considering light input 0208 into the eye 0202, while the lens 0204 provides a similar degree of focusing convergence the distance from the lens 0204 to the retina 0206 is greater, and thus the point of focused image 0210 is disposed in front of the retina 0206 rather than on the surface thereof. Consequently, a viewer with such condition may see the world as appearing out of focus, for example with some degree of myopia.

It is noted that while myopia is addressed in certain examples herein, and reversing, preventing, or slowing progression of myopia may be suitable functions as to be accomplished, embodiments are not limited only to consideration of myopia. At least in principle a variety of eye conditions and concerns may be addressed in similar fashion to examples given herein, and the examples should not be understood to be limiting.

With reference in particular to myopia, however, elongation of the eye as may be seen by comparison of FIG. 01 and FIG. 02 may develop at least in part in response to certain viewing behaviors and conditions for a given subject. For example, routinely spending long periods viewing objects at relatively short distances, e.g., under a few meters (and potentially much less) may lead to physiological processes as may in turn cause axial elongation of the eyeball. In addition, certain types, colors, intensities, etc. of light, variations in contrasts thereof, changes thereof over time, and so forth also may result in such axial elongation. The light input received by the eye may lead to physiological changes in the eye which produce significant changes in eye function, e.g., myopia. However, conversely, certain changes to light input to the eye may diminish or even counteract such potentially harmful physiological changes. The precise properties and distribution of light input as may cause unwanted physiological and/or functional changes, as well as the properties and distribution of light input as may prevent, diminish, or reverse such unwanted physiological and/or functional changes, may be extremely complex and also may be highly variable for different populations, for individuals, and even for a given individual over time. Consequently, approaches as may be fine grained, well controlled, and readily adjusted may prove useful in dealing with myopia and/or other eye health considerations. Fine scale mapping of the retina, and control of light being delivered thereto on a likewise fine scale, even down to an individual pixel by pixel level, may prove more effective than less specific approaches.

Still with reference generally to myopia as an example (though not necessarily limiting embodiments herein only to consideration of myopia), some description of at least one concrete example of concerns as my be considered for treating eye health herein may be illuminating.

Emmetropisation is the process of visual regulation of eye growth towards an optimal refraction (optic). Eye growth is guided by the quality and characteristics of the visual inputs received by the retina. Thus, eye growth can become abnormal (too long/big or too short) due in whole or in part to the particulars of visual inputs as may be received by the eye. For example, if there is a disruption or alteration of the visual inputs received by the retina, or if visual inputs are otherwise atypical, those visual inputs may affect not only the functionality of the eye but the overall shape and size of the eye in addition or instead. More colloquially, what light input the eye receives may directly affect eye health and/or even the physical shape, size, structure, etc. of the eye. (An example of variation in eye size and shape may be noted through a comparison of FIG. 01 and FIG. 02, though this is illustrative and not limiting.)

Thus, the visual environment has a significant role in eye growth, development, and health. Continuing consideration of changes in eye shape/size, as may be understood variations in shape and/or size may affect structural and/or functional features within the eye. For example, the choroid is a layer disposed between the retina and the sclera and is a relay for growth regulatory signals from the retina to the sclera, as may ultimately determine or at least influence eye size and shape. Choroid thickness may change in response to visual inputs received at the retina and/or in the choroid itself (e.g., after passing through the retina). Certain (e.g., typically undesirable) visual inputs/signals may cause the choroid to thin or thicken abnormally, and in such case the choroid may pull the retina backward leading to myopia or forward resulting in hyperopia.

Specifically with regard to myopia, myopia (also referred to commonly as nearsightedness) may result from abnormal and excessive elongation of the eye. In addition to geometric changes to the eye as may affect the manner in which light is focused and perceived, if the eye (or portions thereof) expands and/or distorts, the internal tissues of the eye also may be affected, in particular the retina and choroid may become thin stretched and damaged. Such distortion and/or damage at least potentially may negatively affect vision.

In addition, if myopia occurs in childhood, while the eyes are still developing and may at certain points be growing rapidly, distortion and/or damage may be even more pronounced than if experienced as an adult. Thus consideration of levels of myopia and also ages at which myopia manifests may represent a significant national or global health concern.

Furthermore, in addition to any direct effects of elongating the eye, etc., such as distortion of the retina as the eye changes in shape, such changes may in turn cause, exacerbate, or make more likely certain other eye health issues. For example, elongation of the eye that stretches the retina and choroidal layer may present a significant risk factor for glaucoma, maculopathy, retinal detachment, etc. As may be understood, a greater deformation (as may be correlated with increasingly developed myopia) may correlate with a greater risk and/or increased severity of such eye health effects as may develop as a consequence of such deformation.

Thus, while it may be preferable in at least some instances to halt or even reverse myopia and/or deformation of the eye, changes to the retina and/or choroid, etc., even to decrease or slow the development of myopia within “curing” the problem as such still may be useful. Given that the severity of myopia may vary among individuals, over time within a single individual, etc., interventions as may be readily adjustable over time and/or customizable to groups or even individuals also may be useful.

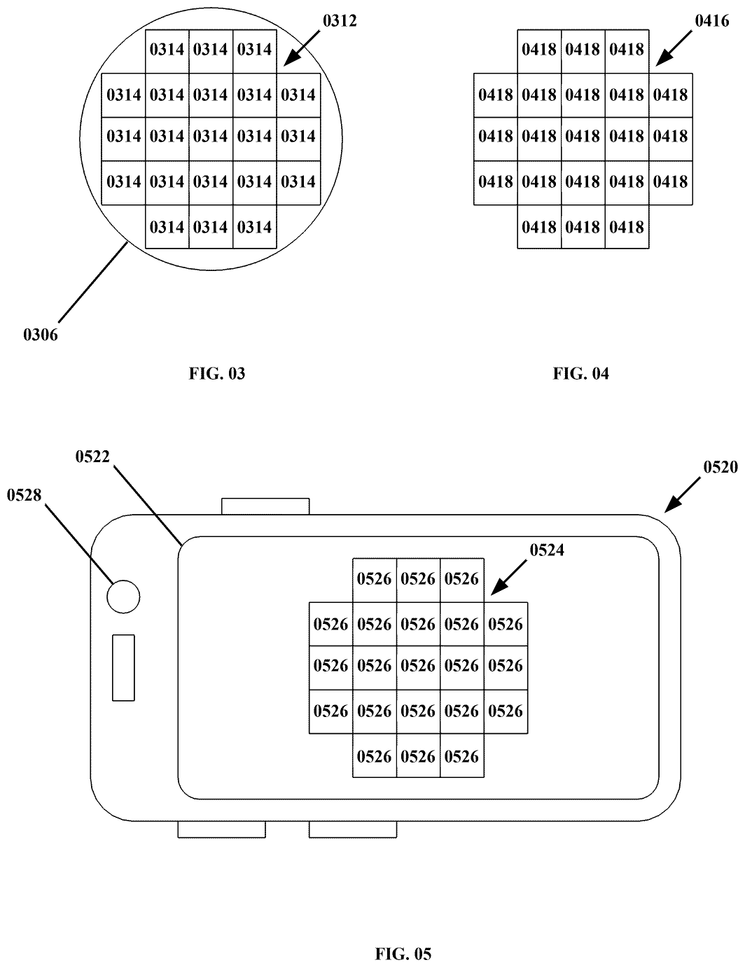

With regard to FIG. 03, FIG. 04, and FIG. 05 collectively, each shows a map; a retinal map 0312 in FIG. 03, a therapeutic light map 0416 in FIG. 04, and a filtering light map 0524 in FIG. 05. While further details are presented subsequently herein, a brief explanation of relationships between maps 0312, 0416, and 0524 may be illuminating. In nonlimiting terms, the retinal map 0312 may be understood as an evaluation of the retina in the sense of “how things are”, e.g., with regard to the health of the retina, how the retina receives light, etc.; the therapeutic light map 0416 may be understood as a prescription for “what should change” in terms of light arriving at the retina in order to accomplish some therapeutic end; and the filtering light map 0524 may be understood as an action of “what is changed” in terms of light output from a display, so that light that does arrive at the actual retina in the various portions of the retinal map 0312 may meet or at least more closely approach the prescriptive therapeutic light map 0416 because of what is applied to the display output via the filtering light map 0524. Thus the three maps are related, and exhibit at least some degree of correspondence, but differ in nature and function. The notion of “what should change” may depend on “how things are”, and the notion of “what is changed” may depend on “what should change”. Ideally, the light output from the display is to be modified so as to better suit the retina, and achieve some therapeutic goal.

Now with reference specifically to FIG. 03, therein is shown a simple representation of a retina 0306 in the form of a circular outline. Again, this is illustrative; in practice a retina 0306 may not be precisely circular, nor is such required. However, as may be seen a retinal map 0312 is presented overlaid onto the retina 0306. The retinal map 0312 as shown includes some 21 individual visual regions 0314 identified therein, though in various embodiments the total number of visual regions 0314 can number in the tens, hundreds, and even thousands. Each visual region 0314 may be evaluated in some manner, such as by ranking health of the retina 0306 in that portion thereof, identifying individual defects or anomalies at that location, measuring some parameter such as thickness of the choroid or visible vascularity or geometry (keeping in mind that the retina typically may not be flat but may conform to the eyeball and so may be concave; though as noted with regard to FIG. 01 and FIG. 02 the shape thereof may vary). Thus, the retinal map 0312 may be understood in some sense as representing a current state of the health, function, geometry, etc. of the retina 0306. In colloquial terms, the retinal map 0312 may be considered a description of “how things are” with regard to eye health (whether considering myopia or some other condition). In terms of the constitution of a retinal map 0312 in practice, one suitable approach (though not necessarily the only such) may be to convert observations/measurements/etc. of the retina 0306 into numerical form, such as an array of pixel values as may be conveniently addressed in quantitative fashion (e.g., by a digital processor, though other approaches may be suitable).

Many possible evaluations may be made regarding the structure, functionality, health, and/or appearance of the retina 0306, and embodiments are not limited with regard to precisely what features or how many features may be considered. Likewise, many possible approaches may be used to conduct such evaluations, and embodiments also are not limited with regard to how evaluations may be carried out. Further, while a particular arrangement of a retinal map 0312 and visual regions 0314 is illustrated in FIG. 03, these are illustrative only. Retinal maps 0312 are not required to utilize any particular number, shape, or arrangement of visual regions 0314, nor is the shape or arrangement of the retinal map 0312 as a whole limited. Moreover, visual regions are not required to be consistent in size, shape, etc; given a higher density of cones in the fovea, for example, it may be useful for certain embodiments to define smaller visual regions 0314 in or near the fovea than elsewhere, etc. In addition, while the retinal map 0312 shown in FIG. 03 covers most but not all of the retina 0306, this is an example only, and different degrees of coverage may be suitable (including full coverage and/or coverage as may extend some distance beyond the retina 0306 itself). Further, while the retina is addressed in this example, broader and/or more comprehensive evaluations of eye health and/or adjustment or other response thereto are not excluded and may be suitable for certain embodiments.

Now with reference to FIG. 04, therein is shown a therapeutic light map 0416 with a number of visual emphasis regions 0418 therein. It is noted that the therapeutic light map 0416 is not presented in combination with a retina or other part of an eye, nor with other physical structures. Rather than being an evaluation of a physical system such as an eye, or being a modification to a device, the therapeutic light map 0416 may be understood as prescriptive for what useful changes may be made to light incoming to a retina, for some therapeutic purpose. Thus, if the retinal map 0312 in FIG. 03 is understood as in some sense a description of the state of the retina 0306 therein, e.g., “how things are”, it may be illuminating to consider the therapeutic light map 0416 in FIG. 04 as representing in some sense “what should change” with regard to light reaching the retina. Considered somewhat differently, the therapeutic light map 0416 may constitute a sort of prescription as to how light should be modified. Each visual emphasis region 0418 may have different parameters, e.g., some may call for a decrease in brightness while others may call for an increase in brightness, some for an increase in color saturation while others for a decrease, etc.

As shown in FIG. 04 the therapeutic light map 0416 exhibits a size, shape, and configuration similar to the retinal map 0312 in FIG. 03. This may be useful for certain embodiments, as such an arrangement facilitates a simple one to one correspondence between the therapeutic light map 0416 and the retinal map 0312. Thus, the map of “what should change” may correlate in very direct fashion with the map of “how things are”. However, such an arrangement is not required, and other approaches may be suitable. While the therapeutic light map 0416 typically may be defined or otherwise established in response to the retinal map 0312—that is, “what should change” may be understood to depend to at least some degree on “how things are”—there may not be such a clear and direct correlation between a therapeutic light map 0416 and a retinal map for any given embodiment (nor is such required). The simple one to one correspondence in FIG. 03 and FIG. 04 (and likewise continuing on in FIG. 05) may be useful for illustrative purposes but is not limiting, and more complex arrangements may be suitable. As a more concrete example, a given therapeutic light map 0416 may exhibit visual emphasis regions 0418 that are different in number, size, configuration, etc. as compared to the visual regions 0314 in the corresponding retinal map 0312.

Now with reference to FIG. 05, therein is shown a display device 0520, as illustrated in the form of a smart phone, though this is an example only and not limiting. As may be seen the display device 0520 includes a display 0522. On the display 0522 may be seen a filtering light map 0524, including a number of display emphasis regions 0526. In the filtering light map 0524, the output of the display is modified from whatever default output that device 0520 might typically deliver. For example, if the device 0520 were being used to watch a video, then whatever portion of the video is in the area of the filtering light map 0524 would be modified in some fashion, e.g., made brighter or dimmer, changed in color or frame rate, etc. The display emphasis regions 0526 may each vary from one another, so that the modification to the default output of the display 0522 typically may not be uniform (though uniform modification is not excluded). The display emphasis applied to each of the various display emphasis regions 0526 within the filtering light map 0524 may be adapted to modify the light from the display 0520 such that light coming from the display 0520 in the area of the filtering light map 0524 may be consistent with what is prescribed by the therapeutic light map 0416 as noted with regard to FIG. 04, so as to deliver a therapeutic result when the light reaches the retina 0306 on the area of the retinal map 0312 as noted with regard to FIG. 03.

Thus, the retinal map 0312, therapeutic light map 0416, and filtering light map 0524 arc distinct maps but cooperate in function. The retinal map 0312 may be considered as diagnostic, mapping the state of the retina 0306; the therapeutic light map 0416 may be considered as prescriptive, specifying what light modifications may be useful given the information about the retina 0306 as compiled into the retinal map 0312; and the filtering light map 0524 may be considered as an intervention, wherein light from some or all of the display 0520 within the area of the filtering light map 0524 may be modified so that such light matches (or at least more closely approximates) what is indicated by the therapeutic light map 0416.

However, the retinal map 0312, therapeutic light map 0416, and filtering light map 0524 do not necessarily have to be identical: in some embodiments they are, in other embodiments they are not. As noted, a one to one correspondence between the retinal map 0312, therapeutic light map 0416, and filtering light map 0524 is not required in terms of the number of regions, the coverage thereof, the shape, etc. In addition, it is noted that the general nature of the retinal map 0312, therapeutic light map 0416, and filtering light map 0524 may be different. The retinal map 0312 typically may be a set of measurements of the retina 0306, e.g., as obtained from an eye examination; the therapeutic light map 0416 may be a set of intended modifications, e.g., as determined from the retinal map 0312 in view of medical information regarding the retina 0306; while the filtering light map 0524 may be a set of specified modifications to be carried out by executable instructions controlling the display, e.g., as determined from the therapeutic light map 0416. Typically though not necessarily, the retinal map 0312, therapeutic light map 0416, and filtering light map 0524 may be numerical information in some form, e.g., pixel values, numerical measurements, etc., however the numerical format and/or approach for each map may not be identical (nor is such required).

As another notable difference, a retinal map 0312, therapeutic light map 0416, and filtering light map 0524 may behave differently in a spatial sense. A retinal map 0312 typically may be stationary at least with respect to the retina 0306 (though the retina 0306 itself may move with the eye). The health and structure of the retina 0306 in the various visual regions 0314 of the retinal map 0312 typically “are what they are”, at least in the short term. Meanwhile, the therapeutic light map 0416 may not necessarily be spatial at all in a strict sense, in that the therapeutic map 0416 may not physically represent or address to any physical object (e.g., may not be a map of any concrete thing). Rather, the therapeutic map 0416 may to some degree be understood as an abstraction of what may be desirable given the information in the retinal map 0312.

Finally, the filtering light map 0524 may be understood to be a map of some portion of a display 0522 at any moment (e.g., pixels thereon, changes to be implemented to the output of those pixels, etc.), however, the filtering light map 0524 may not be stationary with regard to the display 0522 as the retinal map 0312 may be stationary with regard to the retina 0306. Rather, as the subject's eye shifts in orientation, as the subject's head moves (and thus the eye therewith), the portions of the display 0522 as correlate with light that reaches the retina 0306 and/or specific visual regions 0314 thereof also may shift. Thus, the filtering light map 0524 (and the display emphasis regions 0526 thereof) may move from place to place on the display 0522. As the subject shifts their gaze to different parts of the display 0522, different areas of the display 0522, groups of pixels within the display 0522, etc. may be considered part of the filtering light map 0524.

Indeed, portions of the filtering light map 0524 (e.g., individual display emphasis regions 0526) may not merely move but may come in and out of existence, for example, if the subject were to look at a corner of the display 0522 then some portion of their retinal field of view and thus typically also the filtering light map 0524 might spatially extend beyond the edge of the display 0522. If the subject looks away from the display 0522 altogether, the filtering light map 0524 may not exist in a functional sense, insofar as no pixels of the display 0522 may be modified in light output at that time. Thus, the filtering light map 0524 may be understood to be both movable within the confines of the display 0522 and transient with respect to the display 0522 (in that not all of the filtering light map 0524 may apply to the display 0522 at any given moment).

As noted the retinal map 0312 and visual regions 0314 thereof may be determined through various approaches and may include various types of information regarding the retina 0306 in various formats. Embodiments are not limited with regard thereto. Likewise, as noted the therapeutic light map 0416 and visual emphasis regions 0418 may be determined through various approaches, may have various types of information, may be in various formats, etc. Similarly the filtering light map 0524 and display emphasis regions thereof 0526 may be determined through various approaches, may have various types of information, may be in various formats, etc. However, ultimately the filtering light map 0524 should in some form engage with the display 0522; thus the information, format, etc. may typically (though not necessarily) take the form of numerical information as may be handled by a digital processor controlling the display 0522. It may in some sense be useful to understand the chain of retinal map 0312, therapeutic light map 0416, and filtering light map 0524 (linked as previously described herein) as converting retinal health or function into specific pixel values and/or modifications to pixel values.

The form, nature, and extent of such pixel value changes may vary considerably, and is not limited. As noted, emphasis to light output in the various display emphasis regions 0526 may vary between those display emphasis regions 0526, and also may vary over time within a given display emphasis region 0526. Furthermore, even within a given display emphasis region 0526 at a given moment whatever emphasis may be applied to the display 0522, e.g., to pixels therein/pixel values thereof, also may vary. For example, only certain pixels within a display emphasis region 0526 may be selected for emphasis, e.g., only pixels with brightness or color saturation greater than some threshold may be modified. The degree of specificity is not limited, and emphasis applied to the display 0522 may at least in principle vary across the entire display treating every pixel thereof (or even subpixels, e.g., red, green, and blue pixel color channels) independently on a pixel by pixel basis. Fully independent control of several million pixels as may exist in certain displays may require considerable processing power, and such comprehensive and fine grained control is not required, but also is not excluded and may be implemented in certain embodiments.

The particular emphases as may be applied to a display 0522 and/or pixels thereof in the filtering light map 0524 and display emphasis regions 0526 thereof also are not limited. Certain forms of emphasis/change in pixel values/etc. may include but are not limited to those noted below. Also, for any given display emphasis region 0526 at any given moment, none, some, or all pixels of the display 0522 therein may be modified. (Indeed, it is not prohibited that display emphasis regions 0526 be defined as individual pixels.)

Brightness of a display or portions or pixels thereof may be increased or decreased as forms of display emphasis. Contrast on a display among portions or pixels thereof may be increased or decreased. Color saturation of a display or portions or pixels thereof may be increased or decreased, and saturation of individual color channels (e.g., red, green, blue) likewise. Display resolution may be changed, e.g., display resolution may be decreased by grouping adjacent pixels together and treating those pixel groups in terms of display logic and function as larger pixels. A hue of a display or portions or pixels thereof may be altered. A tint may be added to or removed from a display or portions or pixels thereof. The refresh rate of the display or portions or pixels thereof may be increased or decreased as a form of display emphasis. A video frame rate, and/or a speed of an animation and/or other changes in time, may be increased or decreased in a display or portions or pixels thereof. The degree of focus of a display may be increased or decreased in a display or portions or pixels thereof. A blur or “smear” in a display or portions or pixels thereof may be increased or decreased. A degree of sharpness in a display or portions or pixels thereof may be increased or decreased. The presence and/or degree of diffusion in a display or portions or pixels thereof may be increased or decreased as a form of display emphasis. It should be understood that adjusting focus, blur, sharpness, diffusion, etc. may entail changes to groups of pixels rather than individual pixels in certain instances. That is, to “smear” a color transition rather than presenting a sharp edge in color may be accomplished by modifying color values not only along the line but on one or both sides thereof. Addressing pixels by groups in such fashion, or otherwise addressing neighboring pixels for a feature present in a given pixel, is not prohibited and may be present in certain embodiments, even if such embodiments nominally consider/address the output of each pixel individually.

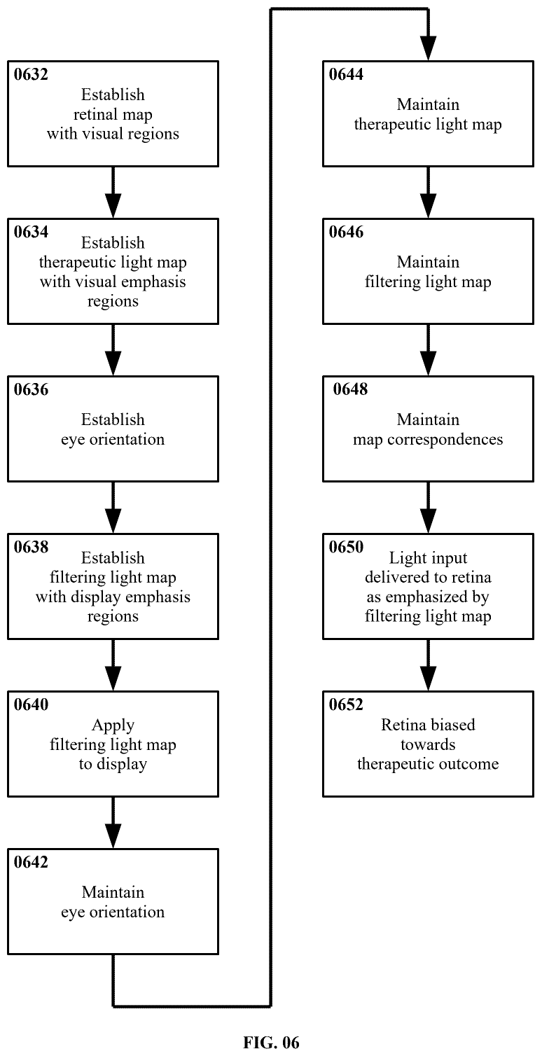

Now with reference to FIG. 06, therein an example method is presented for applying a visual emphasis to provide a therapeutic bias, in flow chart form. It is noted that the arrangement shown is illustrative only, and steps may be combined, subdivided, reordered, etc., to any degree as may be functionally useful and possible.

In FIG. 06, a retinal map of a retina is established 0632, with visual regions therein. Typically though not necessarily, any of a variety of medical and/or diagnostic approaches may be utilized to establish 0632 the retinal map. The particular information in the retinal map as well as the manner by which the map is established may depend to at least some degree on the condition or conditions under consideration, the severity thereof, etc. For example, a retinal map for mild myopia may be established differently and/or include different information than a retinal map for severe myopia, likewise a retinal map for macular degeneration may be established different and/or include different information than a retinal map for myopia.

The term “establishing” as used herein, should be understood broadly. In establishing a retinal map, therapeutic light map, eye orientation, filtering light map, etc., relevant features may be measured, defined, predetermined, calculated, estimated, considered as a convention, determined through medical judgment, loaded onto a processor as data and/or executable instructions, selected by a user, etc. It is noted that such options are not necessarily exclusive, and a given embodiment may utilize more than one. So long as the feature under consideration in some fashion becomes available for the purposes herein, the manner by which a feature may be established is not limited.

Thus, still with reference to FIG. 06, for example in certain embodiments it may be that the retinal map may be established by a medical scan, but alternatively the retinal map may be read from a data store or other storage, e.g., having been created in some fashion previously and now being considered as known information for future use. As another alternative, certain defaults may be assumed regarding the retina rather than the retina being actively mapped as such, for example a simple vision test may suggest the condition of the retina without direct examination. (Such an approach may be expedient in conditions where more comprehensive medical examination is unavailable, for instance.)

Continuing in FIG. 06, a therapeutic light map is established 0634 with visual emphasis regions therein. As described previously herein, typically a therapeutic light map may be understood as prescriptive based on the condition of the retina, thus the therapeutic light map typically (though not necessarily) may be established 0634 based at least in part on information from the retinal map and/or associated scans, diagnostics, etc.

The orientation of an eye (and potentially both eyes, if present) of a subject is established 0636. In colloquial terms, this may be understood as the question of where is the subject looking? Given that both the subject as a whole and a retina thereof may move, change orientation, etc., in order to emphasize a suitable portion of a display so as to deliver modified light to the retina (subsequently herein), it may be necessary to know where the retina is and how the retina is oriented in space. The manner by which eye orientation (and thus typically orientation of the retina) may be determined is not limited. Typically though not necessarily, a display device 0520 such as is illustrated in FIG. 05 may include one or more imagers 0528 such as digital cameras, time of flight sensors, etc. Where not integrated into a display device 0520, orientation of the retina may be established 0636 through the use of separate imagers, use of other sensors, inferred indirectly from the position and/or orientation of the display device (e.g., a smart phone typically may have sensors to determine orientation thereof, and orientation of the smart phone may enable inferences regarding where a subject may be looking on the display thereof), etc. Other approaches also may be suitable.

Continuing in FIG. 06, a filtering light map is established 0638 with display emphasis regions therein. As noted, a filtering light map typically may address the display of a display device, so as to apply emphasis to various individual pixels, display emphasis regions, etc. within the filtering light map so that light from the display on the footprint of the filtering light map may be changed to be more desirable for the subject's retina.

Typically though not necessarily, the retinal map, therapeutic light map, and filtering light map may be established 0632, 0634, and 0638 within or communicated to a digital processor adapted to control a display under consideration. Thus, such maps may include executable instructions, digital information, etc., and certain steps as shown in FIG. 06 may be carried out in or in cooperation with a digital processor.

Still with reference to FIG. 06, the filtering light map is applied 0640 to a display, so as to change light output therefrom by emphasizing features of light output thereof. It is noted that this constitutes a substantive and potentially directly visible change in the operation of the display; functionally, light output from the display is different with the filtering light map applied 0640 than may be the case for that display by default without a filtering light map. While certain steps and elements herein may include or be information in some sense, such as digital information instantiated on a processor, the function of a display device and the generation of light therefrom may be altered as practical matters. (Ultimately light received at the retina likewise may be modified through such emphasis, as well, ideally so as to bias the eye towards a therapeutic end such as opposing the progression of myopia.)

In FIG. 06, eye orientation is maintained 0642. As may be understood eye orientation (and thus retina orientation) with regard to the display in question may vary over time. Thus, ongoing updates thereof may be useful in achieving changes to the light output from proper portions of the display. Further, as may be understood other factors not strictly constituting motion/position/orientation of the eye may be relevant, e.g., if the display is a mobile device and may be held, motion of the subject's hands over time may shift the display. Thus while for clarity discussion herein refers to the orientation of the eye, in practice it should be understood to consider the subject's retina, eye as a whole, head, body, the display, etc., insofar as assuring that the filtering light map is applied 0640 to the right portion of that display at any given time. Similarly, maintaining 0642 eye orientation should be understood to encompass potentially many factors with regard to maintaining a correspondence between the filtering light map on the display and the retina at which light from the filtering light map on the display ultimately may be received and detected.

Continuing in FIG. 06, the therapeutic light map is maintained 0644, e.g., in continuing correspondence with the retina but also at least potentially with regard to other factors. For example, the prescriptive changes to light receiving the retina may not be fixed over time, e.g., the ambient light levels where the subject is located may be relevant as to what modifications should be made to light coming from the display. What may be “good for the retina” at a given time may not be a constant, and may vary with factors such as lighting, the length of time the subject has been using the display, activity levels (e.g., is the subject exercising on a treadmill or sitting at a desk), the content being displayed, and numerous other factors. Consideration of such factors may not be required for all embodiments, but also is not prohibited. Thus, while to at least a certain degree the therapeutic light map may be understood as somewhat stable (at least while the condition of the retina is also stable), some variation therein even on short time scales or in real time may be useful. Thus, while in certain embodiments maintaining 0644 the therapeutic light map may require minimal or no change, in other embodiments changes may be implemented.

The filtering light map is also maintained 0646. For example, as the subject moves their eyes, their head, etc., as the subject moves as a whole with respect to the display or the display itself moves (either by the action of the subject or otherwise), the portion of the display at which the subject is looking—and thus from which light will be received at the retina—may vary over time. As the filtering light map typically may function by being disposed in such an area of the display (though applying filtering to portions of a display that the subject is not looking at is not prohibited), the filtering light map must in essence “move around the screen” over time, based on where the subject is looking. In addition, the filtering light map may change with various factors similar to those noted with regard to step 0644, such as the content of the display at a given time, etc. (Indeed, such changes may be prescribed by the therapeutic light map, so that in at least certain cases maintaining 0644 the therapeutic light map may drive maintaining 0646 of the filtering light map.)

Likewise, insofar as a correspondence must be maintained/may vary due to various changes, the correspondence of the various maps—e.g., the filtering map with the therapeutic map, the therapeutic map with the retinal map—is maintained 0648 in FIG. 06. The filtering light map may be understood to function by altering display output to deliver what is prescribed by the therapeutic light map. Light from the display is modified by the filtering map, which prescribes light to be delivered to the retina according to the retinal map; maintaining correspondence 0648 among maps herein controls to at least some extent the light input that will arrive at the retina (see below).

Light input is delivered 0650 to the retina. More particularly, light input from the display as modified by the filtering light map in accordance with the therapeutic light map is delivered 0650 to the retina as aligned with the retinal map. So long as delivery of such modified light to the retina takes place, the various maps, relationships therebetween, etc. may vary considerably.

Such modified light is delivered to the retina, e.g., by way of converting eye health in the retina to pixel values representing changes in how a display emits light, as controlled by the filtering light map in accordance with the therapeutic light map and in alignment with the retinal map. With delivery of such modified light as described, the retina (and potentially the eye as a whole) may be biased towards a therapeutic outcome 0652. For example considering myopia, control/modification of light as will be delivered from a display to the retina may bias the eye-through various physiological processes, etc.—away from myopia. Thus, control of light input to the retina in such fashion may slow the progression of myopia, halt the progression of myopia, or reverse myopia. On a more gross physical level, such control of light delivered to the retina may result in substantive structural changes to the retina, other tissues in the eye, the overall shape of the eyeball, etc. (For conditions other than myopia, changes as may manifest may differ according to the specific condition in question.)

It is noted that while the retina may be biased 0652 towards a given therapeutic outcome, neither the eye nor specific physiological changes herein may be directly controlled. A visual stimulus is applied herein to the eye (in the form of modified light output from a display), and the eye responds. In essence, the eye “does what it does”, and positive health effects may be useful and/or significant, and may result in physical and/or chemical changes. However, it is not suggested that the application of light as described herein constitutes a more direct intervention or bodily or chemical modification such as surgery or medication.

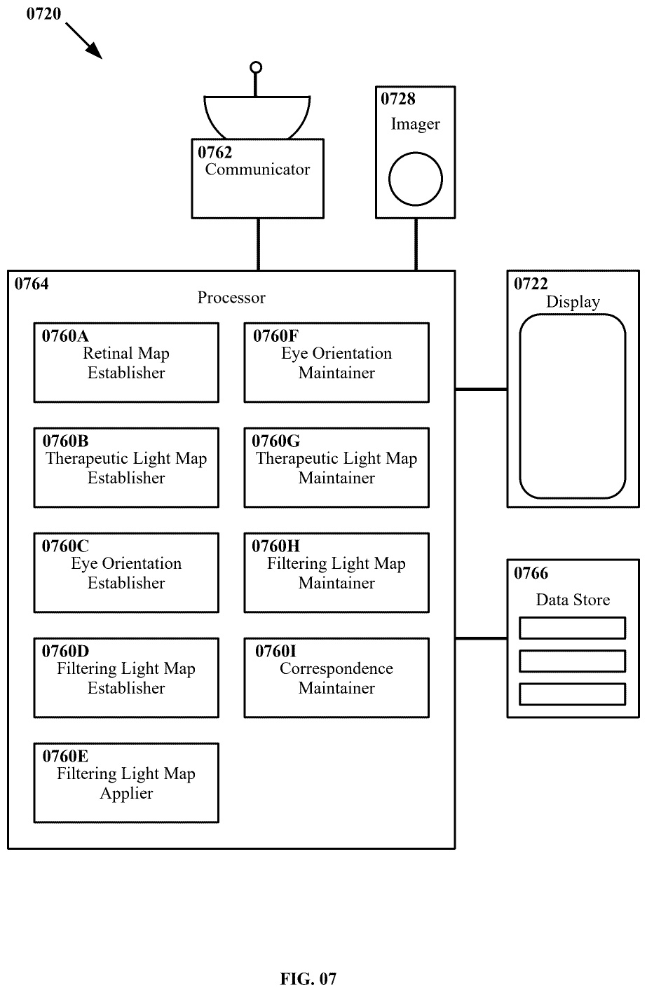

Now with reference to FIG. 07, therein is shown an example illustration of an apparatus as may be suited for applying a therapeutic bias to an eye as described herein. In FIG. 07, a display device 0720 may be seen. The device 0720 may be a smart phone or similar as illustrated previously herein in FIG. 05, but other arrangements may also be suitable, including but not limited to a tablet, laptop computer, desktop computer, television, head mounted display unit, or other video display unit.

As also may be seen, the device 0720 in FIG. 07 is engaged with a display 0722. While the display 0722 is illustrated as being directly connected with a remainder of the display device 0720, this is not required, nor is it required that the display 0722 be integrated into the device 0720 as such. While in certain instances it may be suitable for a display 0722 to be integrated into a display device 0720, as with a smart phone, in other arrangements the display 0722 may be separate, e.g., as a monitor that may be physically distinct from a desktop computer, or as a television that is not even physically connected with some other device (communicating for example through some wireless system, etc.). Other arrangements also may be suitable. It is noted that such provision for either integration or separation may also apply to any or all other elements shown in FIG. 07, as well. While for clarity the display device 0720 is shown as a single unit, this is an example only, and in practice it may be suitable to utilize discrete components without limitation.

Still with reference to FIG. 07, the display device 0720 also is shown to include an imager 0728 such as a digital camera as may be present on smart phones, tablets, laptop computers, etc. (though a discrete imager 0728 also may be suitable, as noted). The imager 0728 is shown in FIG. 07 for illustrative purposes, but not all embodiments necessarily will or must include an imager 0728. As noted previously, an imager 0728 may be useful in performing certain functions such as identifying where a subject is looking, etc., but other arrangements also may be suitable. Similarly, other elements shown as examples in FIG. 07 may be excluded from certain embodiments, and/or additional elements may be part of certain embodiments, so long as functionality is maintained.

The display device 0720 as illustrated also includes a processor 0764. The nature of the processor 0764 is not limited, though typically (but not necessarily) a digital processor may be suitable. Also typically though not necessarily, the processor 0764 may include executable instructions disposed thereon.

The processor 0764 is illustrated in FIG. 07 as having data entities 0760A through 0760I disposed thereon. The data entities 0760A through 0760I shown are adapted to perform various functions (as described subsequently), and may include executable instructions, stored information, etc. FIG. 07 shows the data entities 0760A through 0760I as distinct to individual functions, but in practice such data entities may be combined, subdivided, modified, etc., without limitation. For example, a single mobile device application may include some or all of data entities 0760A through 0760I incorporated therein, with instructions and data therein not necessarily readily distinguishable into discrete parts. So long as the relevant functionality may be accomplished, any number, form, etc. of data entities 0760A through 0760I may be suitable.

With regard to individual data entities 0760A through 0760I, as shown in FIG. 07, each data entity 0760A through 0760I represents a function at least somewhat similar to description previously presented herein. The retinal map establisher 0760A is adapted to establish a retinal map of a retina with visual regions thereof. In certain embodiments the retinal map may be generated as needed, while in other embodiments a retinal map may be established previously and then (for example) loaded from memory, communicated from an eternal source, etc. Thus depending on the embodiment, a retinal map establisher 0760A may facilitate a medical scan, or may simply be a call instruction for stored data, etc. Similarly, other data entities herein may be complex or simple without limit, and data entities may be combined, subdivided, etc.

It is noted that in addition to data entities, other elements, including but not limited to hardware elements, may be utilized in carrying out certain functions, such as imager 0728. For example, the retinal map establisher 0760A may carry out functions at least in part by controlling and/or receiving data from an imager 0728. Likewise, the eye orientation establisher 0760C may carry out functions at least in part by controlling and/or receiving data from an imager 0728. However, neither the retinal map establisher 0760A nor the eye orientation establisher 0760C must necessarily make use of an imager 0728, or other hardware or elements. More broadly, although various data entities 0760A through 0760I are referred to herein as being adapted to perform various functions, such functions need not be performed “in a vacuum” with only the data entities 0760A through 0760I themselves; cooperation with other elements, whether data entities, hardware, etc., may be suitable.

Again with reference to FIG. 07, the therapeutic light map establisher 0760B is adapted to provide a therapeutic light map corresponding with the retinal map, with visual emphasis regions therein. As already described, the therapeutic light map and visual emphasis regions may be considered as prescriptive in relation to the retinal map, e.g., indicating what changes to incoming light may be beneficial to the retina.

Still with reference to FIG. 07, the eye orientation establisher 0760C is adapted to establish an orientation of an eye, and potentially such considerations as specifically an orientation of a retina thereof. As noted previously, eye orientation may include factors other than the eye, as such; for example, the position and/or arrangement of the subject's head, face, the location of a display, etc.

A filtering light map establisher 0760D is adapted to provide a filtering light map, with display emphasis regions therein. As also noted, the filtering light map and display emphasis regions may be understood as being specifications for changing the output of a display, so that light therefrom within the filtering light map may correspond with the therapeutic light map. A filtering light map applier 0760E is in turn adapted to apply the filtering light map to the display 0722, typically though not necessarily by addressing the graphical output from the processor 0764.

An eye orientation maintainer 0760F is adapted to update or otherwise maintain the eye orientation as provided by the eye orientation establisher 0760C, previously mentioned. As an example of combined data entities, in certain embodiments the eye orientation establisher 0760C may itself be adapted to carry out the functions of an eye orientation maintainer 0760F, and the two functions may not imply two distinct data entities. Similar dual functionality may apply to some degree to some or all of data entities 0760G through 0760I in a given embodiment.

Continuing in FIG. 07, a therapeutic light map maintainer 0760G is adapted to maintain the therapeutic light map and visual emphasis regions, e.g., so as to continue to properly define changes in light more suitable to be delivered to the retina. A filtering light map maintainer is adapted to maintain the filtering light map and display emphasis regions, for example in accommodating motion of the eye by shifting the position, configuration, etc. of the filtering light map on the display 0722 so that the proper portion of the display 0722 undergoes modification in light output for delivery to the retina. A correspondence maintainer 0760I is adapted to maintain correspondence among the maps as described herein, e.g., the filtering light map with the therapeutic light map, and the therapeutic light map with the retinal map. Correspondence among such maps as described herein (e.g., map to map, region to region, etc.) to at least some degree controls light input as may be delivered to the retina.

The display device 0720 may also include a data store 0766. The data store 0766 may be adapted to accept, store, and deliver stored data and/or executable instructions, for example so as to support functions within the display device 0720 and in particular (though not necessarily) the data entities 0760A through 0760I. In addition or instead, the data store 0766 also may be adapted to instantiate from storage the data entities 0760A through 0760I onto the processor 0764, and/or to perform other functions such as recording data regarding the subject's behavior and eye condition, responsiveness to therapy, particulars of the maps and regions, etc. Such information also may be (but is not required to be) communicated to some external entity, such as a database, medical professional, the subject themself, etc.

The display device 0720 may also include a communicator 0762, as may be adapted to send and/or receive information between the display device 0720 and one or more external entities. For example, as noted with regard to the data store 0766 information may be communicated to a database, medical professional, etc. In addition, information may be received, such as software updates, new instructions for providing treatment to the subject, etc. Also, in particular it is noted that information may be communicated to other displays, or to the display 0722 of the display device 0720 if the display 0722 is not integral. Other communication also may be suitable.

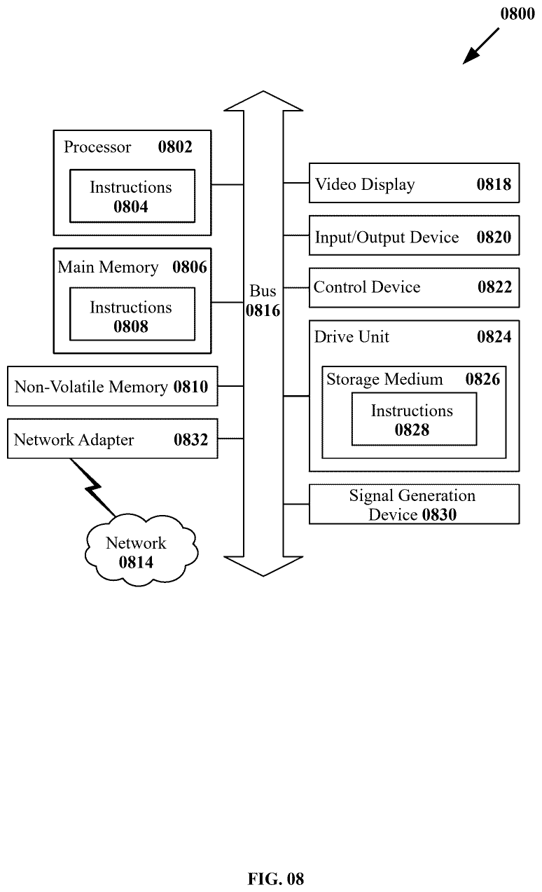

Moving on to FIG. 08, therein is shown a block diagram illustrating an example of a processing system 0800 in which at least some operations described herein can be implemented. The processing system may include one or more central processing units (“processors”) 0802, main memory 0806, non-volatile memory 0810, network adapter 0832 (e.g., network interfaces), video display 0818, input/output devices 0820, control device 0822 (e.g., keyboard and pointing devices), drive unit 0824 including a storage medium 0826, and signal generation device 0830 that are communicatively connected to a bus 0816. The bus 0816 is illustrated as an abstraction that represents any one or more separate physical buses, point to point connections, or both connected by appropriate bridges, adapters, or controllers. The bus 0816, therefore, can include, for example, a system bus, a peripheral component interconnect (PCI) bus or PCI-Express bus, a HyperTransport or industry standard architecture (ISA) bus, a small computer system interface (SCSI) bus, a universal serial bus (USB), IIC (I2C) bus, or an Institute of Electrical and Electronics Engineers (IEEE) standard 1394 bus, also called “Firewire.”

In various embodiments, the processing system 0800 operates as a standalone device, although the processing system 0800 may be connected (e.g., wired or wirelessly) to other machines. In a networked deployment, the processing system 0800 may operate in the capacity of a server or a client machine in a client-server network environment, or as a peer machine in a peer-to-peer (or distributed) network environment.

The processing system 0800 may be a server, a personal computer (PC), a tablet computer, a laptop computer, a personal digital assistant (PDA), a mobile phone, a processor, a telephone, a web appliance, a network router, switch or bridge, a console, a hand-held console, a (hand-held) gaming device, a music player, any portable, mobile, hand-held device, or any machine capable of executing a set of instructions (sequential or otherwise) that specify actions to be taken by the processing system.

While the main memory 0806, non-volatile memory 0810, and storage medium 0826 (also called a “machine-readable medium) are shown to be a single medium, the term “machine-readable medium” and “storage medium” should be taken to include a single medium or multiple media (e.g., a centralized or distributed database, and/or associated caches and servers) that store one or more sets of instructions 0808, 0828. The term “machine-readable medium” and “storage medium” shall also be taken to include any medium that is capable of storing, encoding, or carrying a set of instructions for execution by the processing system and that cause the processing system to perform any one or more of the methodologies of the presently disclosed embodiments.

Still with reference to FIG. 08, in general the routines executed to implement the embodiments of the disclosure may be implemented as part of an operating system or a specific application, component, program, object, module or sequence of instructions referred to as “computer programs.” The computer programs typically comprise one or more instructions (e.g., instructions 0804, 0808, 0828) set at various times in various memory and storage devices in a computer, and that, when read and executed by one or more processing units or processors 0802, cause the processing system 0800 to perform operations to execute elements involving the various aspects of the disclosure.

Moreover, while embodiments have been described in the context of fully functioning computers and computer systems, those skilled in the art will appreciate that the various embodiments are capable of being distributed as a program product in a variety of forms, and that the disclosure applies equally regardless of the particular type of machine or computer-readable media used to actually effect the distribution.

Further examples of machine-readable storage media, machine-readable media, or computer-readable (storage) media include, but are not limited to, recordable type media such as volatile and non-volatile memory devices 0810, floppy and other removable disks, hard disk drives, optical disks (e.g., compact disk read-only memory (CD ROMS), digital versatile disks, (DVDs)), and transmission type media such as digital and analog communication links.

The network adapter 0832 enables the processing system 0800 to mediate data in a network 0814 with an entity that is external to the computing device 0800, through any known and/or convenient communications protocol supported by the processing system 0800 and the external entity. The network adapter 0832 can include one or more of a network adapter card, a wireless network interface card, a router, an access point, a wireless router, a switch, a multilayer switch, a protocol converter, a gateway, a bridge, bridge router, a hub, a digital media receiver, and/or a repeater.

The network adapter 0832 can include a firewall that can, in some embodiments, govern and/or manage permission to access/proxy data in a computer network, and track varying levels of trust between different machines and/or applications. The firewall can be any number of modules having any combination of hardware and/or software components able to enforce a predetermined set of access rights between a particular set of machines and applications, machines and machines, and/or applications and applications, for example, to regulate the flow of traffic and resource sharing between these varying entities. The firewall may additionally manage and/or have access to an access control list which details permissions including for example, the access and operation rights of an object by an individual, a machine, and/or an application, and the circumstances under which the permission rights stand.

As indicated above, the computer-implemented systems introduced here can be implemented by hardware (e.g., programmable circuitry such as microprocessors), software, firmware, or a combination of such forms. For example, some computer-implemented systems may be embodied entirely in special-purpose hardwired (i.e., non-programmable) circuitry. Special-purpose circuitry can be in the form of, for example, application-specific integrated circuits (ASICs), programmable logic devices (PLDs), field-programmable gate arrays (FPGAs), etc.

The foregoing description of various embodiments of the present disclosure has been provided for the purposes of illustration and description. It is not intended to be exhaustive or to limit this disclosure to the precise forms disclosed. Many modifications and variations will be apparent to one skilled in the art. Embodiments were chosen and described in order to best describe the principles of the invention and its practical applications, thereby enabling others skilled in the relevant art to understand the claimed subject matter, the various embodiments, and the various modifications that are suited to the particular uses contemplated.

While embodiments have been described in the context of fully functioning computers and computer systems, those skilled in the art will appreciate that the various embodiments are capable of being distributed as a program product in a variety of forms, and that the disclosure applies equally regardless of the particular type of machine or computer-readable media used to actually effect the distribution.

Although the above Detailed Description describes certain embodiments and the best mode contemplated, no matter how detailed the above appears in text, the embodiments can be practiced in many ways. Details of the systems and methods may vary considerably in their implementation details, while still being encompassed by the specification. As noted above, particular terminology used when describing certain features or aspects of various embodiments should not be taken to imply that the terminology is being redefined herein to be restricted to any specific characteristics, features, or aspects of the invention with which that terminology is associated. In general, the terms used in the following claims should not be construed to limit the claimed invention to the specific embodiments disclosed in the specification, unless those terms are explicitly defined herein. Accordingly, the actual scope of the claimed invention encompasses not only the disclosed embodiments, but also all equivalent ways of practicing or implementing the embodiments under the claims.

The language used in the specification has been principally selected for readability and instructional purposes, and it may not have been selected to delineate or circumscribe the inventive subject matter. Accordingly, the disclosure of various embodiments is intended to be illustrative, but not limiting, of the scope of the embodiments, which is set forth in the following claims.

What is claimed is:

1. A method for biasing an eye of a subject toward a therapeutic outcome, comprising:
determining a retinal map comprising a plurality of visual regions of a retina of said eye;
responsive to said retinal map of said retina, determining a therapeutic light map comprising a plurality of visual emphasis regions, said therapeutic light map comprising a therapeutic correspondence with said retinal map;
determining an eye orientation determination for said eye with respect to a display;
responsive to said therapeutic light map and with respect to said eye orientation, determining a filtering light map for said display comprising a plurality of display emphasis regions, said filtering light map comprising a filtering correspondence with said therapeutic light map, each display emphasis region comprising at least one display emphasis to a display light output from said display;
applying said filtering light map to said display so as to apply said display emphases for said display emphasis regions to said display light output therefrom;
maintaining over time said eye orientation determination for said eye of said subject;
maintaining over time said therapeutic light map and said filtering light map; and
maintaining over time said filtering correspondence of said filtering light map with said therapeutic light map, and maintaining over time said therapeutic correspondence of said therapeutic light map with said retinal map;
wherein:
a light input delivered to said retina from said display as emphasized by said filtering light map corresponds with said therapeutic light map;
said light input delivered to said retina biases said eye towards said therapeutic outcome; and
said therapeutic outcome comprises reversing myopia, preventing myopia, and/or slowing progression of myopia.

2. The method of claim 1, wherein:
at least a portion of said display emphasis regions each address one individual pixel;
such that applying said filtering light map comprises applying said display emphases for said portion of said display emphasis regions to said each one individual pixel.

3. The method of claim 1, wherein:
said display emphases for said display emphasis regions individually address pixels in said display emphasis regions.

4. The method of claim 1, wherein:
said display emphases for said display emphasis regions individually address selected pixels in said display emphasis regions.

5. The method of claim 4, further comprising:
selecting said selected pixels based on a pixel color value thereof.

6. The method of claim 1, wherein:
said display is non-transparent.

7. The method of claim 1, wherein:
determining said retinal map comprises perimetry.

8. The method of claim 1, wherein:
determining said retinal map comprises measuring retinal sensitivity in said plurality of visual regions of said retina.

9. The method of claim 1, wherein:
determining said retinal map comprises performing a Humphrey visual field test.

10. The method of claim 1, wherein:
determining said retinal map comprises determining a deviation from nominal geometry of said retina.

11. The method of claim 1, comprising:
determining an eye geometry model of said eye;
determining said therapeutic light map responsive to said eye geometry model; and
determining said filtering light map responsive to said eye geometry model.

12. The method of claim 1, wherein:
said display emphases for said plurality of display emphasis regions comprise a change to at least one emphasis property comprising:
a brightness;
a contrast;
a saturation;
a resolution;
a hue;
a tint;
a display refresh rate;
a video frame rate;
an animation speed;
a focus;
a blur;
a sharpness; and
a diffusion.

13. The method of claim 1, wherein:
said display emphases for said plurality of display emphasis regions comprise a change to at least one of:
a red channel, a green channel, and a blue channel in a pixel color value;
said red channel but not said green channel and said blue channel in said pixel color value; and
said blue channel but not said red channel and said green channel in said pixel color value.

14. The method of claim 1, wherein:
said therapeutic light map is variable in time.

15. The method of claim 1, wherein:
said filtering light map is variable in time, such that for said display emphasis regions at least a portion of said display emphases thereof are variable in time.

16. The method of claim 1, comprising:
modifying said therapeutic light map responsive to said display light output.

17. The method of claim 1, comprising:
modifying said therapeutic light map responsive to a use of said display by said subject.

18. The method of claim 1, comprising:
modifying said therapeutic light map responsive to a display content of said display.

19. An apparatus for biasing an eye of a subject toward a therapeutic outcome, comprising:
a display;
an eye orientation monitor;
a processor, comprising executable instructions instantiated thereon adapted to:
responsive to a retinal map comprising a plurality of visual regions of a retina of said eye, determine a therapeutic light map comprising a plurality of visual emphasis regions, said therapeutic light map comprising a therapeutic correspondence with said retinal map;
in cooperation with said eye orientation monitor, determine an eye orientation for said eye with respect to said display;
responsive to said therapeutic light map and with respect to said eye orientation, determine a filtering light map for said display comprising a plurality of display emphasis regions, said filtering light map comprising a filtering correspondence with said therapeutic light map, each display emphasis region comprising at least one display emphasis to a display light output from said display;
apply said filtering light map to said display so as to apply said display emphases for said display emphasis regions to said display light output therefrom;
maintain over time said eye orientation determination for said eye of said subject;
maintain over time said therapeutic light map and said filtering light map; and
maintain over time said filtering correspondence of said filtering light map with said therapeutic light map, and maintain over time said therapeutic correspondence of said therapeutic light map with said retinal map;
wherein:
a light input delivered to said retina from said display as emphasized by said filtering light map corresponds with said therapeutic light map;
said light input delivered to said retina biases said eye towards said therapeutic outcome; and
said therapeutic outcome comprises reversing myopia, preventing myopia, and/or slowing progression of myopia.

20. An apparatus for biasing an eye of a subject toward a therapeutic outcome, comprising:
means for determining a retinal map comprising a plurality of visual regions of a retina of said eye;
means for determining a therapeutic light map comprising a plurality of visual emphasis regions, said therapeutic light map comprising a therapeutic correspondence with said retinal map, responsive to said retinal map;
means for determining an eye orientation determination for said eye with respect to a display;
means for determining a filtering light map for said display comprising a plurality of display emphasis regions, said filtering light map comprising a filtering correspondence with said therapeutic light map, responsive to said therapeutic light map and with respect to said eye orientation, each display emphasis region comprising at least one display emphasis to a display light output from said display;
means for applying said filtering light map to said display so as to apply said display emphases for said display emphasis regions to said display light output therefrom;
means for maintaining over time said eye orientation determination for said eye of said subject;
means for maintaining over time said therapeutic light map and said filtering light map; and
means for maintaining over time said filtering correspondence of said filtering light map with said therapeutic light map, and for maintaining over time said therapeutic correspondence of said therapeutic light map with said retinal map;
wherein:
a light input delivered to said retina from said display as emphasized by said filtering light map corresponds with said therapeutic light map;
said light input delivered to said retina biases said eye towards said therapeutic outcome; and
said therapeutic outcome comprises reversing myopia, preventing myopia, and/or slowing progression of myopia.
